(19) United States
(12) Patent Application Publication
(10) Pub. No.: US 2006/0047295 A1
(43) Pub. Date: Mar. 2, 2006

(54) Combination acupuncture/acupressure device and method of application

(76) Inventor: Anna Abramov, Slingerlands, NY (US)

Correspondence Address: GLOBAL PATENTS, Suite 120, 838 W. Long Lake Road, Bloomfield Hills, MI, 48302, US

(21) Appl. No.: 11/212,968

(22) Filed: Aug. 25, 2005

Related U.S. Application Data
(60) Provisional application No. 60/606,339, filed on Sep. 1, 2004.

(51) Int. Cl. A61B 17/34

(52) U.S. Cl. 606/189

(57) ABSTRACT

The present invention provides a device and related methods for the self-administered provision of acupuncture-like benefits without the insertion of needles into one's skin or the risk of contraction of a disease from unsterilized needles.

### CROSS-REFERENCE TO RELATED APPLICATIONS

This application claims the benefits of a Provisional Application No. 60-606,339 filed Sep. 1, 2004 under 35 U.S.C.§119(e).

### FIELD OF THE INVENTION

The present invention relates generally to therapeutic devices and methods, and more particularly, to a combination acupuncture/acupressure device and related methods.

### BACKGROUND OF INVENTION

Acupuncture has been practiced in traditional Chinese medicine for thousands of years. By inserting thin needles below the surface of the skin, acupuncture therapy promotes healing and improves various functions of the body. In some instances, electrical or thermal energy are applied to the needles after insertion.

In traditional Chinese medicine, the theory behind acupuncture therapy is that regularly patterned channels (meridians) or energy (qi) are located throughout the body in association with the nerve, circulatory, and immune systems. Disruptions in the normal flow of energy cause corresponding disruptions in these systems and may lead to associated disorders and diseases. These disruptions in the normal flow of energy can be relieved by the insertion of thin needles into the skin at specific points in the body. Hundreds of such specific points are known to exist along the meridians of the body.

However, many individuals are adverse to the insertion of needles into their skin. In addition, it is possible to transmit hepatitis and other infectious diseases through the use of unsterilized needles. As a result, many individual who might benefit from acupuncture therapy will not engage in it due to an aversion to needles or the possibility of contacting a disease.

Furthermore, acupuncture therapy may only be effective when administered by one trained in such therapy. Often, engaging the services of such an individual is not possible, due to limited availability or prohibitive expense. Therefore, a need exists for devices and methods for the provision of the benefits of acupuncture and acupressure therapies without the attendant insertion of needles or risk of disease contraction and which are capable of self-administration.

### SUMMARY OF INVENTION

The present invention provides a device and related methods for the self-administered provision of acupuncture-like benefits without the insertion of needles into one's skin or the risk of contraction of a disease from unsterilized needles.

A first aspect of the invention provides a device for the delivery of a therapeutic effect comprising a base member and at least one pointed member, wherein application of the at least one pointed member to the skin of an individual provides a therapeutic effect.

A second aspect of the invention provides a device for the delivery of therapeutic effect comprising a mat and a plurality of base members each having a base and at least one pointed member emanating from the base wherein application of the at least one pointed member to the skin of an individual provides a therapeutic effect.

A third aspect of the invention provides a method for delivering a therapeutic effect to an individual comprising the steps of providing g a device having at least one base member, each base member having at least one pointed member, applying the device to the individual such that the at least one pointed member contacts the individual's skin, applying a pressure to the at least one base member, thereby increasing the contact area between the at least one pointed member and the individual's skin.

The foregoing and other features of the invention will be apparent from the following more particular description of embodiments of the invention.

### BRIEF DESCRIPTION OF THE DRAWINGS

The embodiments of this invention will be described in detail, with reference to the following figures, wherein like designations denote like elements, and wherein:

FIG. 1 is a perspective view of the first embodiment of a base member of the invention.

FIG. 2 is a top view of the first embodiment of a base member of FIG. 1.

FIG. 3 is a top view of a second embodiment of the base member of the invention.

FIG. 4 is a top view of a mat with a plurality of the first embodiment of the base member installed thereon.

FIG. 5 is a top view of an alternate embodiment of the mat of FIG. 4.

FIG. 6 is a cross-sectional view of a mat and a pressurizing device according to the invention.

### DETAILED DESCRIPTION OF THE INVENTION

The present invention provides a device and related methods for the self-administered provision of acupuncture-like benefits without the insertion of needles into one's skin or the risk of contraction of a disease from unsterilized needles.

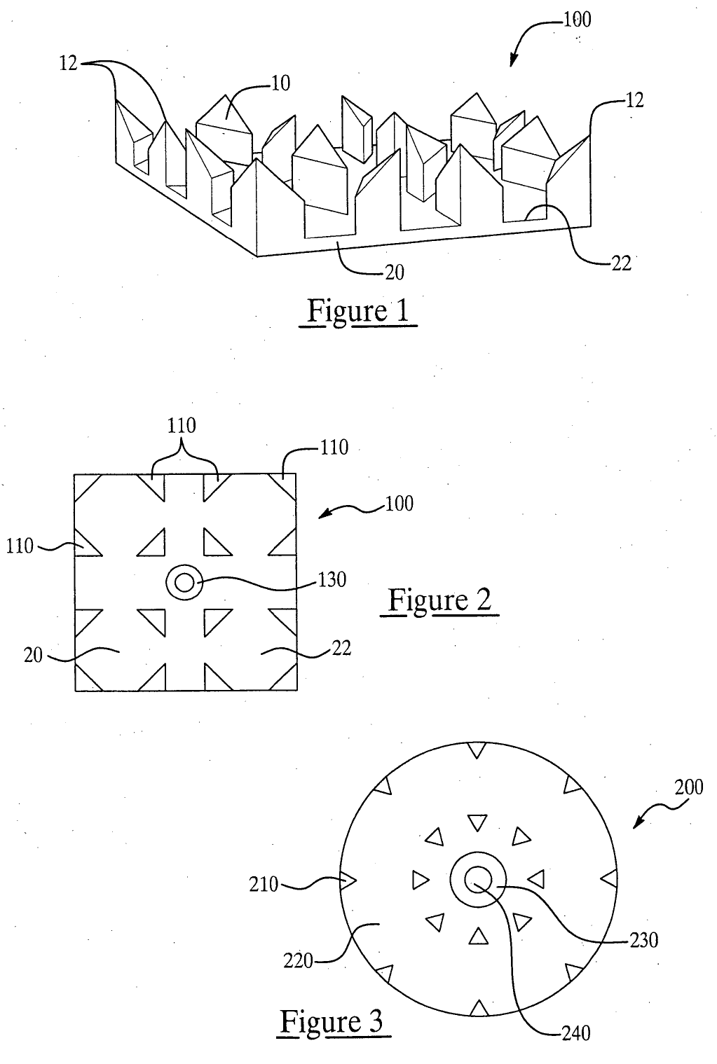

Referring initially to FIG. 1, a base member 100 is shown having a base 20 and a plurality of pointed members 10. Base 20 has a least one substantially flat surface 22 upon which a plurality of pointed members 10 may be present. The plurality of pointed members 10 and base 20 may be separate members or may be integrally formed from a single piece of material. Acceptable materials for the plurality of pointed members 10 and base 20 include, for example, plastics, metals, ceramics, and wood. The base 20 may have a suitable thickness of about 1 mm., or in the range between about 0.1 mm and about 10 mm. Each of the plurality of pointed member 10 may have a suitable height of 6 mm, or in the range of between about 1 mm and about 20 mm.

Referring now to FIG. 2, a plurality of pointed members 110 is arranged in a pattern of squares on a surface 22 of the base 20, although any number of different patterns are possible. The pattern of squares of 100 is possible due to a cross-sectional shape of pointed members 110, which, as depicted, is triangular. However, any other cross-sectional shape may be utilized, including, for example, circular, ovoid, square, and rectangular, provided that a tip 12 pointed away from the base 20, terminates in at least one point. In fact, pointed members 110 may have multiple cross-sectional shapes along its height through different planes. The tip 12 of pointed member 110 should be small enough as to at least partially depress the skin of an individual, thereby providing a therapeutic effect, but yet no too small as to penetrate the skin. For instance, the diameter of the tip 12 should be at least 0.1 mm.

Similarly, the base 20 may have any one of a number of cross-sectional shapes. As depicted in FIGS. 1 and 2, base 20 is substantially square. Other shapes are allowable and within the scope of the invention, including, for example, circular, ovoid, and rectangular. FIG. 3 shows another embodiment of the base member 200, wherein base 220 is substantially circular. The plurality of pointed members 210 are arranged in a circular pattern positioned at various radii from the center 240 of base 220. each of the plurality of pointed members 210 are again depicted as having a triangular cross-sectional shape.

In some embodiments of the invention, it is desirable to attach a plurality of base members 100, 200 to a mat or similar structure in order to provide a therapeutic effect over a larger area of an individual's skin. In doing so, it is necessary to attach the base member 100, 200, to a substrate, or a mat 340. Accordingly, an attachment device 130, 230 may be incorporated into or applied to base member 120, 220. Suitable attachment device include, for example, adhesives, rivets, pins, clips, screws, staples, thread, and bolts. The attachment device 130, 230 chosen will depend, to some degree, on the materials of the base member 120, 220 and the mat 340.

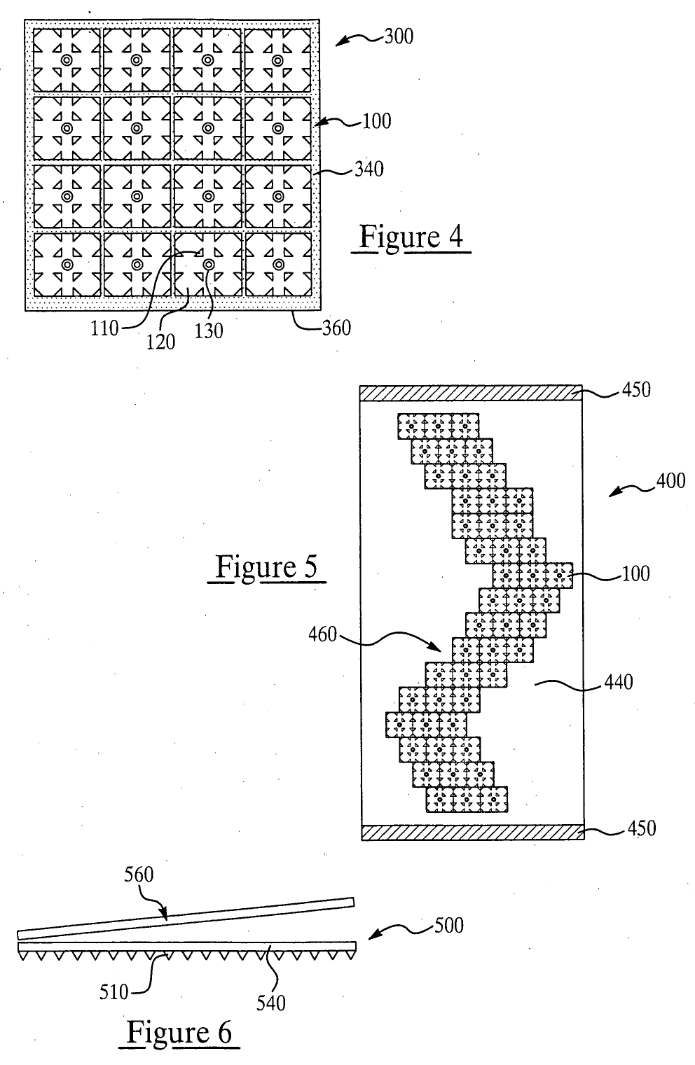

FIG. 4 depicts a therapeutic sheet 300, wherein individual base members 100 are attached to a mat 340. Each base member comprises a plurality of pointed members 110, a base 120, and an attachment device 130. Mat 340 may be cut into any shape and size capable of accommodating the number of base members 100 desired, including, for example, square, rectangular, circular and ovoid. Mat 340 may similarly be formed of any one of a number of materials, depending on the application. For instance, where therapeutic sheet 300 is to be applied to a curved or uneven area of an individual's skin, it is desirable that mat 340 be flexible. In other applications, it may be desirable that mat 340 be rigid. Accordingly, mat 340 may be formed, for example, in a natural or synthetic fabric, a plastic, a rubber, metal, wood or combination of such materials.

In order to enhance the acupuncture-like therapeutic effects of the invention, it may be beneficial to apply the device of the invention in a pattern corresponding to an acupuncture meridian. FIG. 5 depicts a therapeutic sheet 400, wherein a plurality of base members 100 have been attached to mat 440 in a pattern 460 corresponding to an acupuncture meridian. In order to aid the application of therapeutic sheet 400 to certain areas of the body, a securing device 450 may be incorporated into mat 440. Securing device 450 is particularly useful in securing therapeutic sheet 400 around the arm, leg, or torso of a user. Suitable securing devices 450 include, for example, hook-and-loop fasteners, snaps, buttons and clips.

FIG. 6 depicts a cross-sectional view of a therapeutic sheet 500 having a pressurizing device applied thereto. Pressurizing device 560 increases pressure on a surface of mat 540, enhancing the contact of the plurality of pointed members 501 with an individuals skin. Pressurizing device 560 may be formed integral with the therapeutic sheet 500 or may be a separate device. Any object capable of applying pressure to mat 540 may be used. However, the pressurizing device 560 should not provide so much pressure as to cause a puncture of an individual's skin by the plurality of pointed member 510. As with mat 540, where therapeutic sheet 500 is to be applied to a contoured or uneven area of an individual's skin, the pressurizing device 560 is preferably flexible. Accordingly, the pressurizing device 560 may be a heavy cloth or fabric or a bag-like device containing individual weighted members, similar to a bean bag (not shown). In some situations, it may be advantageous to combine thermal therapy with the application of the therapeutic sheet 500. In such situations, the pressurizing device 560 may incorporate a heating/cooling device, such as hot or cold water. Such an embodiment further provides thermal therapy in the course of providing pressure therapy.

When used, a device of the invention, such as the therapeutic sheet 300 in FIG. 4, is placed against an individual's skin with the plurality of pointed members 110 contacting the skin. Pressure is then applied to a back surface 360 of mat 340 not containing the plurality of base members 100, which transfers pressure to the plurality of base 20 and from there to the plurality of pointed members 110, thereby enhancing the contact area of the pointed members 110 with the individual's skin and providing a therapeutic effect similar to that of acupuncture therapy.

While the present invention has been described in conjunction with the specific embodiments outlined above, it is evident that many alternatives, modifications and variations will be apparent to those skilled in the art. Accordingly, the embodiments of the invention as set forth above are intended to be illustrative, not limiting. Various changes may be made without departing from the spirit and scope of the invention as defined in the following claims.

What is claimed is:

1. A device for providing a therapeutic effect to a human body comprising:
a base member; and
at least one pointed member situated on said base member, said at least one pointed member having at least one tip portion pointing away from said base member;
wherein said at least one pointed member is applied to the skin of a human body such that said at least one tip portion of said at least one pointed member intimately contacts the skin of the human body to provide a therapeutic effect.

2. The device of claim 1, wherein said base member and said at least one pointed member are integrally formed.

3. The device of claim 1, wherein said at least one pointed member having a height between about 1 mm and about 20 mm.

4. The device of claim 1, wherein said at least one pointed member has a triangular cross-section at a base portion.

5. The device of claim 1, wherein said at least one tip portion having a diameter of at least about 0.1 mm.

6. The device of claim 1, wherein said base member has a planar surface formed in a shape selected from the group consisting of a parallelogram, a trapezoid, a polygon, an oval, and a triangle.

7. The device of claim 1, further comprising a fastening device for attaching said base member to a mat.

8. The device of claim 7, wherein said fastening device is at least one member selected from the group consisting of an adhesive, a rivet, a pin, a clip, a screw, a staple, a thread, and a bolt.

9. A device for delivery of a therapeutic effect to a human body comprising:
a mat; and
a plurality of base members situated on said mat, each of said plurality of base members having at least one pointed member thereon; and
wherein application of said at least one pointed member to the skin of a human body provides a therapeutic effect.

10. The device of claim 9, wherein the plurality of base members is arranged on said mat in a pattern corresponding to at least one meridian known in the field of acupuncture.

11. The device of claim 9, further comprising a pressurizing device for application to a back surface of the mat, thereby increasing the pressure of said at least one pointed member against the skin of a human body.

12. The device of claim 11, wherein said pressurizing device is a member containing a quantity of a liquid.

13. The device of claim 11, wherein said pressurizing device further comprising a heating/coding device for providing a thermal effect to the skin of said human body.

14. The device of claim 9, further comprising a securing device for holding said mat against a portion of said human body.

15. The device of claim 14, wherein said securing device is at least one of a hook-and-loop fastener, snaps, buttons, and clips.

16. A method for delivering a therapeutic effect to a human body comprising the steps of:
providing a device having at least one base member, each of said at least one base member having at least one pointed member thereon;
applying said device to a human body such that said at least one pointed member intimately contacts a skin of said human body; and
applying a pressure to the at least one base member, thereby increasing the contact area between said at least one pointed member and the skin of said human body without piercing said skin.

17. The method of claim 16, further comprising the step of forming said at least one base member in a shape selected from the group consisting of a parallelogram, a trapezoid, a polygon, an oval, and a triangle.

18. The method of claim 16, further comprising the step of forming a base portion of said at least one pointed member in a triangular cross-section.

19. The method of claim 16, further comprising the step of applying said pressure by a pressurizing means.

20. The method of claim 16, further comprising the step of arranging said plurality of base members in a swivel pattern.
